SEQUENCE LISTING (13 sequences)

SEQ ID NO: 1 - DNA, 10 bp, Artificial
  Synthetic
tttttttttt

SEQ ID NO: 2 - DNA, 50 bp, Artificial
  Synthetic
tttttttttttttttttttttttttttttttttttuuuuuuuuuuuuuuu

SEQ ID NO: 3 - DNA, 25 bp, Artificial
  synthetic
ttttttttttttttttttttttttt

SEQ ID NO: 4 - DNA, 12 bp, Artificial
  synthetic
tttttttttttt

SEQ ID NO: 5 - DNA, 14 bp, Artificial
  synthetic
tttttttttttttt

SEQ ID NO: 6 - DNA, 10 bp, Artificial
  synthetic
tttttttttt

SEQ ID NO: 7 - DNA, 50 bp, Artificial
  synthetic
tttttttttttttttttttttttttttttttttttttttttttttttttt

SEQ ID NO: 8 - RNA, 25 nt, Artificial
  synthetic
  misc_feature (22)..(25): n is a, c, g, or u
ccuuggcacccgagaauuccannnn

SEQ ID NO: 9 - DNA, 29 bp, Artificial
  synthetic
  misc_feature (1)..(3): n is a, c, g, or t
nnngatcgtcggactgtagaactctgaac

SEQ ID NO: 10 - DNA, 50 bp, Artificial
  synthetic
aatgatacggcgaccaccgagatctacacgttcagagttctacagtccga

SEQ ID NO: 11 - DNA, 63 bp, Artificial
  synthetic
caagcagaagacggcatacgagatcgtgatgtgactggagttccttggcacccgagaattcca

SEQ ID NO: 12 - DNA, 26 bp, Artificial
  synthetic
ctagcagcctgacatcttgagacttg

SEQ ID NO: 13 - DNA, 22 bp, Artificial
  synthetic
gccttggcacccgagaattcca

(19) United States
(12) Patent Application Publication
(10) Pub. No.: US 2018/0265912 A1
(43) Pub. Date: Sep. 20, 2018

(54) MODIFIED 3' REGION EXTRACTION AND DEEP SEQUENCING OF POLYDENYLATION SITES AND POLY(A) TAIL LENGTH ANALYSIS

(71) Applicant: Rutgers, The State University of New Jersey, New Brunswick, NJ (US)

(72) Inventors: Bin Tian, Woodcliff Lake, NJ (US); Dinghai Zheng, Harrison, NJ (US)

(21) Appl. No.: 15/853,055

(22) Filed: Dec. 22, 2017

Related U.S. Application Data
(63) Continuation of application No. PCT/US17/37927, filed on Jun. 16, 2017.
Application No. PCT/US17/37927 is a continuation-in-part of application No. 14/240,514, filed on Jul. 24, 2014, now abandoned.
(60) Provisional application No. 62/350,909, filed on Jun. 16, 2016.
(60) Provisional application No. 61/526,672, filed on Aug. 23, 2011.
(60) Provisional application No. 61/526,676, filed on Aug. 23, 2011.

(51) Int. Cl. C12Q 1/6806; C07H 21/02; C40B 50/00; C12Q 1/6813; C12Q 1/6876; C12N 9/22; C12N 9/00; C07H 19/06

(52) U.S. Cl. CPC C12Q 1/6806; C07H 21/02; C40B 50/00; C12Q 1/6813; C12Q 1/6876; C12N 9/22; G06F 19/22; C07H 19/06; C12Q 2525/173; C12Q 2600/166; C12Q 2600/158; C12N 2310/11; C12N 2310/3181; C12N 9/93

(57) ABSTRACT

The present invention relates to modified 3′ region extraction and deep sequencing of polyadenylated RNA to identify a poly(A) site in a reference, as well as to calculate poly(A) tail length.

### I. CROSS-REFERENCE TO RELATED APPLICATIONS

The present application is a continuation of International Patent Application Serial No. PCT/US17/37927, filed on Jun. 16, 2017, which claims the benefit of priority under 35 U.S.C. § 119(e) of U.S. Provisional Patent Application Ser. No. 62/350,909 filed Jun. 16, 2016. The present application is also a continuation-in-part of U.S. Nonprovisional application Ser. No. 14/240,514, filed Jul. 24, 2014, the U.S. National Phase of International Application Serial No. PCT/US12/52122, filed Aug. 23, 2012, which claims the benefit of priority under 35 U.S.C. § 119(e) of U.S. Provisional Patent Application Ser. No. 61/526,672, filed Aug. 23, 2011 and U.S. Provisional Patent Application Ser. No. 61/526,676, filed Aug. 23, 2011. The entire disclosures of the applications noted above are incorporated herein by reference.

### II. STATEMENT REGARDING FEDERAL FUNDING

This invention was made with government support under grant number GM084089 awarded by the National Institute of Health (NIH). The United States government has certain rights in the invention.

### III. FIELD OF THE INVENTION

The present invention relates to methods and kits relating to modified 3′ region extraction and deep sequencing of polyadenylated (poly(A)+) RNA to measure RNA abundance and identify poly(A) sites in a reference, e.g. a reference gene, genome, or genomic database, identify 3′ end of RNA, e.g. for gene expression analysis, as well as methods and kits to calculate poly(A) tail length.

### IV. BACKGROUND OF THE INVENTION

Studies in recent years have revealed that most mRNA genes in eukaryotes contain multiple cleavage and polyadenylation sites, or poly(A) sites, resulting in alternative cleavage and polyadenylation (APA) isoforms with different coding sequences (CDS) and/or variable 3′ untranslated regions (3′UTRs). Dynamic APA regulation has been reported in different tissue types, cancers, cell proliferation/differentiation, development, and response to extracellular stimuli. In addition, a sizable fraction of long non-coding RNA genes also display APA, whose consequences are yet to be fully appreciated.

While APA can be analyzed with data from microarray, serial analysis of gene expression (SAGE) or RNA-seq, these techniques were not specifically designed to identify poly(A) sites, leading to incomplete analysis. These methods are particularly ineffective when poly(A) sites of different isoforms are located close to one another. However, isoforms using different poly(A) sites within a short window have been shown to have quite different metabolisms, making it necessary to examine APA isoforms with precise tools. A number of deep sequencing methods have been developed to specifically sequence the 3′ end of transcripts. These methods can not only identify poly(A) sites but also examine gene expression. Most methods use primers containing the oligo(dT) sequence for reverse transcription (RT). While efficient, oligo(dT) can prime at internal A-rich sequences, leading to false poly(A) site identification. This issue is usually addressed computationally by eliminating putative poly(A) sites in A-rich regions. However, this approach not only cannot guarantee full elimination of false positives caused by internal priming, but can also discard bona fide poly(A) sites.

Some sequencing methods are not affected by internal priming, including 3P-seq (poly(A)-position profiling by sequencing) and 3′READS (3′ region extraction and deep sequencing), e.g. as disclosed in US 2014/0329700, incorporated by reference in its entirety. However, such methods require a large amount of input RNA (25 μg RNA typically used by 3′READS and 20-70 μg RNA recommended for 3P-seq). In addition, poly(A) sites located in a long stretch of As cannot be effectively identified by these methods because the short poly(A) tail left after RNase H digestion can be completely aligned to the A-stretch sequence, leaving no additional A's as evidence of the poly(A) tail. Furthermore, previous studies (Chang et al. (2014) Mol Cell 53, 1044-1052 and Subtelny et al. (2014) Nature 508, 66-71, both references hereby incorporated by reference in their entireties) have indicated that different poly(A) sites can have different poly(A) tail lengths, which are physically relevant to mRNA stability and translation. However, these previous methods to sequence the poly(A) tail are cumbersome or require special sequencing machines. Accordingly, there is a need for improved methods of polyadenylation mapping and a need for methods to reliably and accurately calculate poly(A) tail length.

### V. SUMMARY OF THE INVENTION

In some embodiments, the present invention is directed to a method of obtaining a sample comprising polyadenylated (“poly(A)+”) RNA. In some embodiments, the method comprises obtaining a sample comprising poly(A+) RNA. In some embodiments, the method comprises contacting the sample with a capture oligonucleotide to create isolated poly(A)+ RNA; fragmenting the non-poly(A) region of isolated poly(A)+ RNA to create fragmented poly(A)+ RNA; eluting the fragmented poly(A)+ RNA from the capture oligonucleotide to create free poly(A)+ RNA. In some embodiments, the method comprises ligating the free poly(A)+ RNA to a 5′-adapter to create 5′-adapter ligated poly(A)+ RNA. In some embodiments, the method comprises contacting the 5′-adapter ligated poly(A)+ RNA with a chimeric oligonucleotide (“CO”) to create CO-bound 5′-adapter ligated poly(A)+ RNA. In some embodiments, the CO consists of a protection region (“PR”) and a digestion region (“DR”), wherein the PR is between 5 and 15 nucleotides in length, the first nucleotide of the PR is an antisense oligonucleotide which is capable of binding to a poly(A) tail of poly(A)+ RNA, at least one of every three nucleotides in the PR is an antisense oligonucleotide which is capable of binding to the poly(A) tail of poly(A)+ RNA, and the remaining nucleotides in the PR consist of deoxythymidine, wherein the DR consists of 5 to 50 deoxythymidines, and wherein the orientation of the CO is 5′-DR-PR-3′. In some embodiments, the method comprises incubating the CO-bound 5′-adapter ligated poly(A)+ RNA with RNase H to partially remove the poly(A) tail of CO-bound 5′-adapter ligated poly(A)+ RNA to create bound 5′-adapter-ligated partially digested poly(A)+ RNA sequencing candidates. In some embodiments, the method comprises eluting the bound 5′-adapter ligated partially digested poly(A)+ RNA sequencing candidates from an undigested CO segment to create free 5′-adapter ligated partially digested poly(A)+ RNA sequencing candidates. In some embodiments, the method comprises ligating the free 5′-adapter ligated partially digested poly(A)+ RNA sequencing candidates to a 3′-adapter to create fully ligated poly(A)+ RNA sequencing candidates. In some embodiments, the ligating occurs in the presence of a crowding agent. In some embodiments, the method comprises reverse transcribing the fully ligated poly(A)+ RNA sequencing candidates to create corresponding single-stranded (ss) complementary DNA (cDNA) sequences. In some embodiments, the method comprises amplifying the corresponding ss DNA sequences to create a cDNA library. In some embodiments, the method comprises aligning at least one sequence from the cDNA library to a reference. In some embodiments, positive alignment against the reference together with more than or equal to two (≥2) unaligned terminal nucleotides from the poly(A) sequence indicates a poly(A) site in the reference. In some embodiments, the poly(A) site identifies the 3′ end of the poly(A)+ RNA in the reference. In some embodiments, the method further comprises the relative abundance of the poly(A)+ RNA to determine a gene expression profile.

In some embodiments, the antisense oligonucleotide comprises at least one of a locked nucleic acid, 2′-O-methyl RNA (OMe), 2′-O-methoxy-ethyl RNA (MOE), N3′-P5′ phosphoramidate (NP), cyclohexene nucleic acid (CeNA), 2-fluoro-arabino nucleic acid (FANA), phosphoroamidate morpholino (PMO), tricyclo-DNA, peptide nucleic acid (PNA), and combinations thereof. In some embodiments, the antisense oligonucleotide comprises a locked nucleic acid, and the locked nucleic acid comprises locked deoxythymidine (+T).

In some embodiments, the present invention is directed to a method of calculating poly(A) tail length. In some embodiments, the method comprises obtaining a sample comprising poly(A)+ RNA. In some embodiments, the method comprises adding a predetermined amount of RNA having identical sequences but with variable poly(A) tail lengths to the sample. In some embodiments, the method comprises contacting the sample with a capture oligonucleotide to create isolated poly(A)+ RNA. In some embodiments, the method comprises eluting the poly(A)+ containing RNA from the capture oligonucleotide by one of a mild wash (“Mild Wash” sample) or a stringent wash (“Stringent Wash” sample) to create free poly(A)+ RNA. In some embodiments, the method comprises ligating the free poly(A)+ RNA to a 5′-adapter to create 5′-adapter ligated poly(A)+ RNA. In some embodiments, the method comprises contacting the 5′-adapter ligated poly(A)+ RNA with a chimeric oligonucleotide (“CO”) to create CO-bound 5′-adapter ligated poly(A)+ RNA. In some embodiments, the CO consists of a protection region (“PR”) and a digestion region (“DR”), wherein the PR is between 5 and 15 nucleotides in length, the first nucleotide of the PR is an antisense oligonucleotide which is capable of binding to a poly(A) tail of poly(A)+ RNA, at least one of every three consecutive nucleotides in the PR is a an antisense oligonucleotide which is capable of binding to a poly(A) tail of poly(A)+ RNA, and the remaining nucleotides in the PR consist of deoxythymidine, wherein the DR consists of 5 to 50 deoxythymidines, and wherein the orientation of the CO is 5′-DR-PR-3′. In some embodiments, the method comprises incubating the CO-bound 5′-adapter ligated poly(A)+ RNA with RNase H to partially remove the poly(A) tail of the poly(A)+ RNA to create bound 5′-adapter ligated partially digested poly(A)+ RNA sequencing candidates. In some embodiments, the method comprises eluting the bound 5′-adapter ligated partially digested poly(A)+ RNA sequencing candidates from an undigested CO segment to create free 5′-adapter ligated partially digested poly(A)+ RNA sequencing candidates. In some embodiments, the method comprises ligating the free 5′-adapter ligated partially digested poly(A)+ RNA sequencing candidates to a 3′-adapter to create fully ligated poly(A)+ RNA sequencing candidates. In some embodiments, the ligating occurs in the presence of a crowding agent. In some embodiments, the method comprises reverse transcribing the fully ligated poly(A)+ RNA sequencing candidates to create corresponding single-stranded (ss) DNA sequences. In some embodiments, the method comprises amplifying the corresponding ss DNA sequences to create a cDNA library. In some embodiments, the method comprises aligning at least one sequence from the cDNA library to a reference, wherein positive alignment against the reference gene or genome and existence of more than or equal to two unaligned terminal nucleotides indicates a poly(A) site in the reference. In some embodiments, the method comprises calculating poly(A) tail length of the poly(A)+ RNA sequencing candidates. In some embodiments, calculating poly(A) tail length of the poly(A)+ RNA sequencing candidates comprises calculating the log 2(ratio) of the read number from the “Stringent Wash” sample to that from the “Mild Wash” sample. In some embodiments, the poly(A) site identifies the 3′ end of the poly(A)+ RNA in the reference. In some embodiments, the method further comprises the relative abundance of the poly(A)+ RNA to determine a gene expression profile.

In some embodiments, the antisense oligonucleotide comprises at least one of a locked nucleic acid, 2′-O-methyl RNA (OMe), 2′-O-methoxy-ethyl RNA (MOE), N3′-P5′ phosphoramidate (NP), cyclohexene nucleic acid (CeNA), 2-fluoro-arabino nucleic acid (FANA), phosphoroamidate morpholino (PMO), tricyclo-DNA, peptide nucleic acid (PNA), and combinations thereof. In some embodiments, the antisense oligonucleotide comprises a locked nucleic acid, and the locked nucleic acid comprises locked deoxythymidine (+T).

In some embodiments, the capture oligonucleotide is bound to magnetic beads. In some embodiments, the chimeric oligonucleotide is immobilized on beads or other solid surfaces. In some embodiments, the first ligating step utilizes T4 RNA ligases. In some embodiments, the second ligating step utilizes T4 RNA ligases. In some embodiments, the protection region (PR) of the chimeric oligonucleotide (CO) consists of alternating locked/unlocked deoxythymidines. In some embodiments, the protection region (PR) of the chimeric oligonucleotide has a formula (+TT)₅ (SEQ ID NO: 1). In some embodiments, the chimeric oligonucleotide (CO) is linked to one or more secondary molecules. In some embodiments, the secondary molecule is biotin. In some embodiments, the 3′-adapter is a 5′-adenylated and 3′-blocked 3′ adapter. In some embodiments, the crowding agent is one of polyethylene glycol (PEG), Ficoll, Dextran, hexamine cobalt chloride, ovalbumin, hemoglobin, bovine serum albumin, and combinations thereof. In some embodiments, the crowding agent is polyethylene glycol (PEG). In some embodiments, the aligning step utilizes BLAST alignment. In some embodiments, the reference is a genome. In some embodiments, the reference is a gene. In some embodiments, the reference is a database. In some embodiments, the sample comprises a biological sample. In some embodiments, the sample comprises an environmental sample. In some embodiments, the poly(A)+ RNA in the sample comprises RNA that is modified to include a poly(A) tail region. In some embodiments, the poly(A) tail region is synthesized by contacting the RNA with poly(A) polymerase in vitro.

In some embodiments of the present invention, the invention is directed to an oligonucleotide. In some embodiments, the oligonucleotide is a chimeric oligonucleotide (“CO”). In some embodiments, the CO consists of a protection region (“PR”) and a digestion region (“DR”), wherein the PR is between 5 and 15 nucleotides in length, the first nucleotide of the PR is an antisense oligonucleotide which is capable of binding to a poly(A) tail of poly(A)+ RNA, at least one of every three consecutive nucleotides in the PR is a antisense oligonucleotide which is capable of binding to a poly(A) tail of poly(A)+ RNA, and the remaining nucleotides in the PR consist of deoxythymidine, wherein the DR consists of 5 to 50 deoxythymidines, and wherein the orientation of the CO is 5′-DR-PR-3′. In some embodiments, the antisense oligonucleotide comprises at least one of a locked nucleic acid, 2′-O-methyl RNA (OMe), 2′-O-methoxy-ethyl RNA (MOE), N3′-P5′ phosphoramidate (NP), cyclohexene nucleic acid (CeNA), 2-fluoro-arabino nucleic acid (FANA), phosphoroamidate morpholino (PMO), tricyclo-DNA, peptide nucleic acid (PNA), and combinations thereof. In some embodiments, the antisense oligonucleotide comprises a locked nucleic acid, and the locked nucleic acid comprises locked deoxythymidine (+T).

In some embodiments of the present invention, the invention is directed to a kit. In some embodiments, the kit includes a chimeric oligonucleotide (“CO”). In some embodiments, the CO consists of a protection region (“PR”) and a digestion region (“DR”), wherein the PR is between 5 and 15 nucleotides in length, the first nucleotide of the PR is an antisense oligonucleotide which is capable of binding to a poly(A) tail of poly(A)+ RNA, at least one of every three consecutive nucleotides in the PR is an antisense oligonucleotide which is capable of binding to a poly(A) tail of poly(A)+ RNA, and the remaining nucleotides in the PR consist of deoxythymidine, wherein the DR consists of 5 to 50 deoxythymidines, and wherein the orientation of the CO is 5′-DR-PR-3′. In some embodiments, the antisense oligonucleotide comprises at least one of a locked nucleic acid, 2′-O-methyl RNA (OMe), 2′-O-methoxy-ethyl RNA (MOE), N3′-P5′ phosphoramidate (NP), cyclohexene nucleic acid (CeNA), 2-fluoro-arabino nucleic acid (FANA), phosphoroamidate morpholino (PMO), tricyclo-DNA, peptide nucleic acid (PNA), and combinations thereof. In some embodiments, the antisense oligonucleotide comprises a locked nucleic acid, and the locked nucleic acid comprises locked deoxythymidine (+T).

In some embodiments, the kit includes RNase III. In some embodiments, the kit includes RNase H. In some embodiments, the kit includes T4 RNA ligases. In some embodiments, the kit includes at least one crowding agent. In some embodiments, the crowding agent is one of polyethylene glycol (PEG), Ficoll, Dextran, hexamine cobalt chloride, ovalbumin, hemoglobin, bovine serum albumin, and combinations thereof. In some embodiments, the crowding agent is polyethylene glycol (PEG). In some embodiments, the kit includes instructions for use. In some embodiments, the present invention is directed to use of the kits. In some embodiments, the use of the kit comprises use for identification of a poly(A) site in a reference. In some embodiments, the use of the kit comprises use for identification of a 3′ end of a poly(A)+ RNA. In some embodiments, the use of the kit comprises use for gene expression analysis.

### VI. BRIEF DESCRIPTION OF THE FIGURES

FIG. 1A: Top, schematic showing digestion of the poly(A) tail annealed to the T₃₅U₁₅ (SEQ ID NO: 2) oligo by RNase H. The A's hybridized to deoxythymidines (T's) are digested by RNase H whereas those to uridines (U's) are not. RNase H digestion is indicated by a lightening symbol. The T₃₅U₁₅ oligo contains a 5′ biotin group which can bind to streptavidin-coated beads. Bottom, autoradiography showing digestion products of an RNA molecule containing 60 A's (named A60) by different amounts of RNase H (U/reaction is units per reaction) using the T₃₅U₁₅ (SEQ ID NO: 2) oligo. MW, molecular weight markers (sizes indicated). Number of remaining A's in digestion products are indicated, which were calculated based on the molecular weight markers. FIG. 1B: Top, schematic showing digestion of the poly(A) tail annealed to the T₁₅(+TT)₅ (SEQ ID NO: 3) oligos. +T, being identified as locked deoxythymidine, as described herein. Bottom, autoradiography showing digestion products of A60 by 0.5 unit of RNase H with different oligos. Number of remaining A's in the digestion products is indicated. FIG. 1C: Autoradiography showing binding of RNAs with different numbers of consecutive As to the biotin-T₁₅(+TT)₅ (SEQ ID NO: 3) attached to magnetic beads after washing with buffers containing different concentrations of NaCl and formamide. A60, A15, A10, and A5 have different numbers of consecutive A's and are otherwise the same. FIG. 1D: Quantification of the amount of A15 and A10 bound to biotin-T₁₅(+TT)₅ (SEQ ID NO: 3) relative to A60 in each washing condition based on the data in FIG. 1C.

FIG. 2A: Ligation protocols tested. In protocol A, ligation with 3′ and 5′ adapters were carried out sequentially in the same tube. The 5′ adapter is an RNA oligo with hydroxyl groups at both 5′ and 3′ ends, and the 3′ adapter is a 5′-adenylated DNA oligo with a 3′ blocker (ddC). In protocol B, 5′ adapter ligation was carried out first without PEG, and the ligation product was purified using oligo(dT)₂₅ beads and then ligated to the 3′ adapter in the presence of PEG. FIG. 2B: Autoradiography showing ligation products using different ligation protocols. MW, molecular weight markers (sizes indicated). Schematics of ligation products and their expected sizes are shown on the right. The percent of product shown below the image is based on the amount of RNA with both 5′ and 3′ adapters relative to that of input RNA. FIG. 2C: Bar plot showing the fractions of raw reads with inserts <23 nt from the 3′READS libraries prepared with ligation protocol A with (left) or without (right) PEG and with ligation protocol B. FIG. 2D: Autoradiography showing the effect of PEG on 3′ adapter ligation. RNAs corresponding to the bands are indicated. Percent of product shown below the image is based on the amount of RNA with ligated 3′ adapter relative to that of input RNA. FIG. 2E: Autoradiography showing the effect of PEG on 5′ adapter ligation. Percent of product shown below the image is based on the amount of RNA with ligated 5′ adapter to that of input RNA.

FIG. 3A: A 3′READS+ protocol incorporating optimized RNase H digestion and ligation steps. AAAₙ, poly(A) tail; Aₙ, shortened poly(A) tail. 5′ adapter, 3′ adapter, random sequences in the adapters (3×Ns), and index region in PCR primer are indicated. FIG. 3B: Schematic showing different parts of a raw read generated by 3′READS+. FIG. 3C: Number of 5′ Ts in reads from 3′READS+ and 3′READS. Only the reads mapped to poly(A) sites are shown. FIG. 3D: Sequencing quality of the bases after 5′Ts. Left, schematic showing the analyzed region. Right, the average Quality Score (QS) of the next 20 bases after 5′Ts are shown. QS>28 is usually considered high quality whereas <20 low quality. FIG. 3E: Left, scatter plots comparing log 2(UPM) of transcript between libraries with different amounts of input RNAs. Right, table summarizing correlations between different samples. UPM, UMI Per Million. UMI was based on cleavage site location, number of 5′Ts, RNA fragment size, and the three random nucleotides from the 3′ adapter, as shown in FIG. 3B. Only transcripts with >5 unique PASS reads were used for the plots. Pearson correlation coefficient (r) is indicated in each graph and the table. FIG. 3F: As in FIG. 3E, except that samples from different batches were compared.

FIG. 4A: Schematic showing alignment of a PASS read with an A-stretch region. FIG. 4B: Number of 5′ Ts aligned to the genome for PASS reads using data from HeLa cells. FIG. 4C: Nucleotide profiles around the A-stretch and other poly(A) sites. FIG. 4D: An example gene (Thap2) with an A-stretch poly(A) site. Top, gene structure as shown in the UCSC genome browser. Middle, UPM values for poly(A) sites of Thap2. Three alternative poly(A) sites are indicated. Bottom, sequence surrounding the A-stretch poly(A) site. The AUUAAA polyadenylation signal and the A-stretch region are indicated. Several 3′ READS+ reads are shown to indicate additional As used as evidence for the poly(A) tail. FIG. 4E: Assessment of APA rate in HeLa cells using different numbers of PASS reads and different isoform abundance cutoffs. The plateaued value (51% genes with APA) with the 5% isoform abundance cutoff is indicated by a horizontal line, and two vertical lines indicate 7 and 14 million reads, which gave rise to 49% and 51% APA rates, respectively.

FIG. 5A: schematics of 3′READS+PAT. Top, barcoded spike-in A-tail rulers with known poly(A) tail sizes. The barcodes can be sequenced and used for RNA identification. Bottom, procedures of 3′READS+PAT. Cellular RNA is mixed with spike-in A-tail rulers and bound to oligo(dT)25 beads. The beads were split into two aliquots washed three times with either mild or stringent wash buffer. The beads were used separately as inputs of 3′READS+. The spike-in A-tail rulers were identified by their barcodes (located immediately upstream of the polyA site which allows identification of the sequence) and were used to predict poly(A) tail sizes of cellular RNAs. FIG. 5B The log 2-transformed S/M (RPM after stringent wash/RPM after mild wash) ratios of spike-in RNAs correlate very well with their tail sizes. RPM is read per million of mapped reads per sample.

### VII. DETAILED DESCRIPTION OF THE INVENTION

The present invention covers methods for identifying (e.g. mapping) poly(A) sites in a given reference, such as a reference gene, genome, or database, methods for analyzing poly(A) tail length, and compositions and kits for performing such methods. The methods for identifying polyadenylation sites in a reference may be referred to as 3′READS+, which stands for “modified 3′ region extraction and deep sequencing.” The methods for calculating poly(A) tail length may be referred to as 3′READS+PAT, which is a modification/extension of the core 3′READS+ method as described herein, but particularly adapted to calculate poly(A) tail length (PAT).

3′READS+ may be conceptually divided into a first “module” and a second “module.” The first module is modified for 3′READS+PAT, but the second module is generally consistent between 3′READS+ and 3′READS+PAT, except for the addition of a step at the end of the method to calculate poly(A) tail length, discussed in greater detail infra. The first module of 3′READS+ may be thought of containing steps directed to steps directed to obtaining a sample, isolating poly(A)+ RNA from the sample, fragmenting the poly(A)+ RNA sample, and then elution/recovery of the free poly(A)+ RNA sample. The second module of 3′READS+ contains steps directed to ligating the free poly(A)+ RNA sample with a 5′ adapter, contacting the ligated poly(A)+ RNA with a chimeric oligonucleotide (“CO”) containing locked deoxythymidine as described herein, incubating/partially digesting the bound poly(A)+ RNA with RNase H, eluting the partially digested poly(A)+ RNA from the chimeric oligonucleotide, ligating the poly(A)+ RNA with a 3′ adapter, optionally in the presence of a crowding agent, reverse transcribing the fully ligated poly(A)+ RNA into single stranged (ss) DNA, amplifying the ssDNA to create a cDNA library, and then aligning the cDNA to a reference (e.g. gene, genome, or genomic database) to identify the poly(A) sites in the reference.

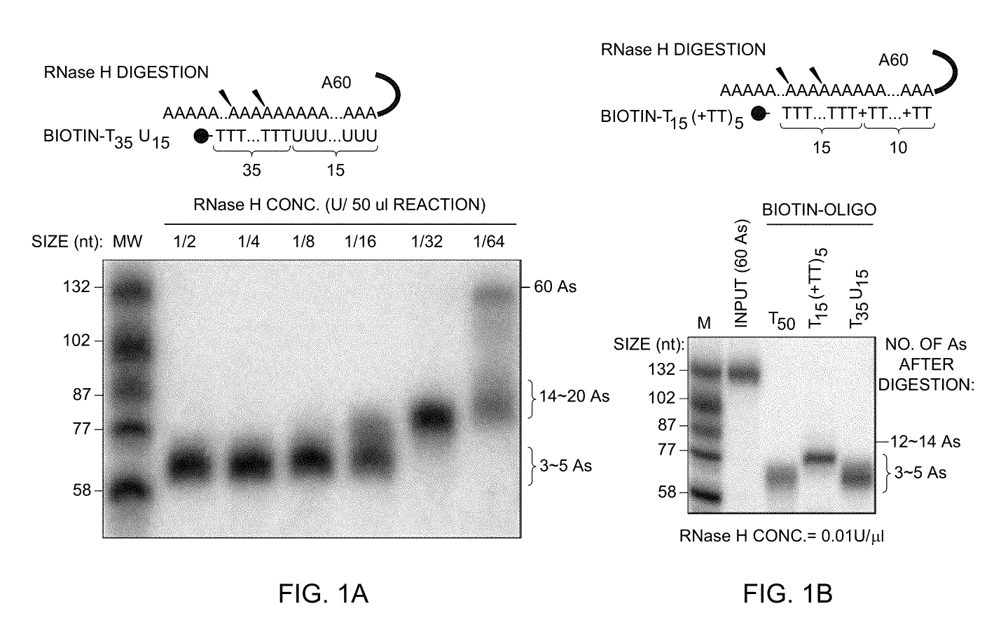

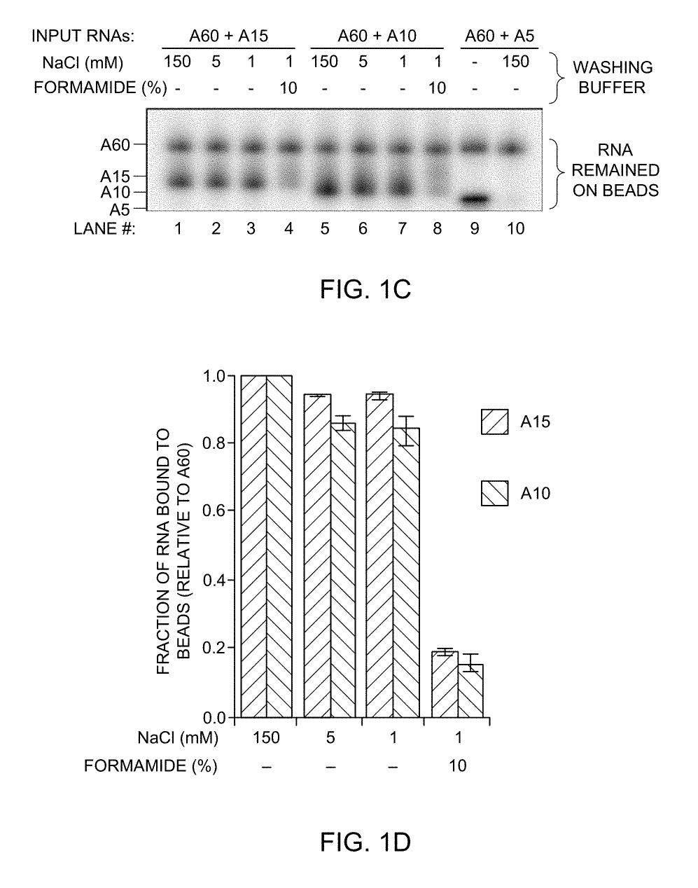

3′READS+ is examined in Example 1, FIGS. 1-4, and generally comprises the following steps. First, a sample containing total RNA is obtained, e.g. a biological sample, although the sample is not necessarily such and may be, for example, an environmental sample. Next, RNA containing a poly(A) tail region (poly(A)+ RNA) is isolated from the total RNA to create isolated poly(A)+ RNA. The isolated poly(A)+ RNA may be mRNA, although any linear branch of RNA is suitable for this purpose, it need not be transcribed from a DNA template. This isolating step may be accomplished by using a capture oligonucleotide, for example a capture oligonucleotide having a repeat string of deoxythymidines, such as, for example, a repeat string of 25 deoxythymidines, although a range from about 15 to about 35 would work as well. The capture oligonucleotide may be bound to, e.g. magnetic beads, or to cellulose columns, and other similar structures known to one of ordinary skill in the art. Next, the non-poly(A) region of isolated poly(A)+ RNA is fragmented using, for example, RNase III to create fragmented poly(A)+ RNA, although other suitable methods include using a metal base or metal ion solutions. This step may occur in a buffer, and such buffers are known to one of ordinary skill in the art, for example but explicitly not limited to Tris-Cl, NaCl, MgCl₂, DTT, or combinations thereof. An exemplary step utilizes each of the buffers in combination at 37° C. for 15 minutes, although variations on this method are acceptable and are considered within the scope of this invention. After fragmentation, any unbound RNA fragments are washed away, e.g. (but not necessarily) by a stringent wash, leaving behind only fragmented poly(A)+ RNA. One of ordinary skill in the art will appreciate what constitutes appropriate stringent wash conditions, namely that the stringent wash must include a buffer that could wash off RNA molecules that have non-specific interactions with the capture oligonucleotide, but not the poly(A)+ RNA. Next, the fragmented poly(A)+ RNA is eluted from the capture oligonucleotide, e.g. by using a TE buffer (Tris-Cl, EDTA, pH 7.5) at 65 or 70° C., to create free poly(A)+ RNA, although the elution may occur by other methods known to one of skill in the art, and recovered, e.g. by precipitation. Other buffers will be known to one of ordinary skill in the art. Precipitation may be accomplished by means known in the art, e.g. by ethanol.

After recovery of the free poly(A)+ RNA, the free poly(A)+ RNA undergoes a first ligation step to a 5′-adapter, e.g. a heat-denatured 5′-adapter to create 5′-adapter ligated poly(A)+ RNA. This first ligation step may utilize a T4 RNA ligase, e.g. T4 RNA ligase 1. Next, the 5′-adapter ligated poly(A)+ RNA is bound to a chimeric oligonucleotide (“CO”) that serves to protect the poly(A) tail of the poly(A)+ RNA from complete digestion by RNase H, creating CO-bound 5′-adapter ligated poly(A)+ RNA. The CO is comprised of two primary components, a first region that directly protects the poly(A) tail from digestion by RNase H detailed herein as the “protection region” (“PR”), and a second region that is subjection to cleavage and digestion by RNase H, detailed herein as the “digestion region” (“DR”). The CO is organized as 5′-DR-PR-3′. The PR of the CO in an exemplary embodiment includes an alternating sequence of locked (+T) and unlocked (T) deoxythymidines, however it is not limited as such. For example, any of the following antisense oligonucleotides would be acceptable: a locked nucleic acid (e.g. locked deoxythymidine (+T)), 2′-O-methyl RNA (OMe), 2′-O-methoxy-ethyl RNA (MOE), N3′-P5′ phosphoramidate (NP), cyclohexene nucleic acid (CeNA), 2-fluoro-arabino nucleic acid (FANA), phosphoroamidate morpholino (PMO), tricyclo-DNA, peptide nucleic acid (PNA), and combinations thereof. These antisense oligonucleotides are examined in more detail in Chan et al. (2006) Clin Exp Pharmacol Physiol.; 33(5-6):533-40, hereby incorporated by reference in its entirety.

The primary functional limitation is that the antisense oligonucleotides must be capable of binding to the poly(A) tail of poly(A)+ RNA. This is because RNase H is capable of digesting a bond between deoxythymidine (T) and adenosine (A), but not capable of digesting the bond formed between an antisense oligonucleotide, for example, a locked nucleic acid such as locked deoxythymidine (+T) and adenosine (A). Example 1 infra utilizes (+TT)₅ (SEQ ID NO: 1) as an exemplary embodiment of a PR. However, this particular PR is only exemplary as others may be designed and utilized for this purpose. For example, a PR that has an antisense oligonucleotide, e.g. locked deoxythymidine (+T) appearing only every three nucleotides as opposed to alternating locked/unlocked deoxythymidine, e.g. (+TTT+T)₃ (SEQ ID NO: 4) or (+TTT+T)₂(T+T)₃ (SEQ ID NO: 5) or even (+T)₁₀ (SEQ ID NO: 6) would be suitable for the invention. While not wishing to be bound by theory, this is because RNase H needs at least three consecutive non-locked nucleotides for digestion. Thus, introducing an antisense oligonucleotide, such as locked deoxythymidine (+T), at least once every three nucleotides in the PR allows the PR to effectively prevent digestion by RNase H. One of ordinary skill in the art will thus understand that there are many possible PR sequences of various lengths that are within the scope of this invention. Notwithstanding the foregoing description, for quality control issues, the total length of the PR should be between 5 to 15 (inclusive) nucleotides total in length, and preferably although explicitly not necessarily is around 10 nucleotides in length. Second, by definition the PR must always begin with an antisense oligonucleotide, e.g. locked deoxythymidine (+T), as the introduction of such into the CO is what separates the PR from the DR, although after introduction of the first antisense oligonucleotide, as previously noted, the requirement is only that there be one antisense oligonucleotide per every three nucleotides in the PR.

As discussed herein, the total length of the PR is largely what determines the length of the resultant bound 5′-adapter ligated poly(A)+ RNA sequencing candidates after digestion with RNase H (discussed infra). Although while not wishing to be bound by theory, ultimately the resultant poly(A)+ RNA sequence will have a few additional nucleotides beyond that of the PR in length presumably due to structural hindrance. As opposed to the PR, which may vary in composition as detailed herein, the DR consists of a string of deoxythymidine (T). As further opposed to the PR, the length of the DR is much more variable, and can be between, for example, 5 and 50 (inclusive) nucleotides in length. Example 1 infra utilizes (T)₁₅ as an exemplary DR, however other lengths as described may be utilized and still be within the scope of this invention. The chimeric oligonucleotide may be linked to a secondary molecule, e.g. in exemplary embodiments, the chimeric oligonucleotide is linked to biotin and is subsequently able to be immobilized by streptavidin (such as in streptavidin-coated beads or a coasted substrate), although such is not necessary and only serves to enhance the method.

After binding of the 5′-adapter ligated poly(A)+ RNA to the CO to create CO-bound 5′-adapter ligated poly(A)+ RNA, the CO-bound 5′-adapter ligated poly(A)+ RNA is preferably washed with a buffer, and is incubated with RNase H, preferably in presence of RNase H buffer. Exemplary conditions include those in Example 1 infra, e.g. 37° C. for 30 min. with Tris-Cl, NaCl, MgCl₂, and/or DTT. As detailed herein, the RNase H serves to digest an unprotected region of the CO-bound 5′-adapter ligated poly(A)+ RNA, i.e. the region (if any) of the poly(A)+ RNA bound to the DR of the PO, thus leaving behind only 5′-adapter ligated poly(A)+ RNA that is bound to the PR (plus potentially 2-3 additional nucleotides that are not digested by RNase H, if any). This step thus creates bound 5′-adapter ligated partially digested poly(A)+ RNA sequencing candidates. Next, the bound 5′-adapter ligated partially digested poly(A)+ RNA sequencing candidates are eluted from the CO by an elution buffer, e.g. NaCl, EDTA, and/or TWEEN 20, although the elution may occur by other methods known to one of skill in the art, and recovered, e.g. by precipitation, thus creating free 5′-adapter ligated partially digested poly(A)+ RNA sequencing candidates. Precipitation may be accomplished by means known in the art, e.g. by ethanol.

Once recovered, the free 5′-adapter ligated partially digested poly(A)+ RNA sequencing candidates are then ligated for a second time, this time to a 3′-adapter, e.g. a 5′-adenylated 3′-blocked 3′-adapter, which is preferably but not necessarily a heat-denatured adapter. This creates fully ligated poly(A)+ RNA sequencing candidates. This second ligation may utilize, for example, truncated T4 RNA ligase 2. The second ligation step utilizes a crowding agent, preferably polyethylene glycol (PEG), although one of ordinary skill in the art will appreciate there are a wide variety of crowding agents that could be used. Some non-limiting examples that are considered within the scope of the invention include, but are explicitly not limited to, Ficoll, Dextran, Hexamine cobalt chloride, ovalbumin, hemoglobin, bovine serum albumin, and other such compounds. Surprisingly, utilization of a crowding agent such as PEG greatly increases ligation efficiency of the free 5′-adapter ligated partially digested poly(A)+ RNA sequencing candidates to the 3′-adapters, although it has been further discovered that utilization of a crowding agent such as PEG results in inter-molecular ligation of the free poly(A)+ RNAs. Thus, the present disclosure has split the ligation steps into a first ligation step to a 5′-adapter prior to digestion by RNase H, and then into a second ligation step to a 3′-adapter that is in the presence of a crowding agent, e.g. PEG, post digestion by RNase H. Such methodology greatly increases yield and quality of the resultant fully ligated poly(A)+ RNA sequencing candidates over prior methods that ligated free poly(A)+ fragments to 5′ and 3′-adapters after digestion by RNase H, without the presence of a crowding agent. After formation of the fully ligated poly(A)+ RNA sequencing candidates, the fully ligated poly(A)+ RNA sequencing candidates may be precipitated and recovered.

The fully ligated poly(A)+ RNA sequencing candidates are then reverse transcribed to create corresponding single-stranded (ss) DNA sequences, and then subjected to amplification, e.g. by PCR, to create a double-stranded cDNA library. One of ordinary skill in the art will be familiar with the creation of a cDNA library, see Example 1 infra for a working example. After creation of the cDNA library, DNA sequences from the cDNA library may undergo sequence alignment against a known or mapped reference, e.g. by BLAST alignment, although other such local alignment tools exist and are known to one of ordinary skill in the art, such as Bowtie, Bowtie 2.0, and similar programs. Alignment hits against the mapped reference, e.g. a reference genome, reference database, reference gene, etc., and existence of more than or equal to two (≥2) unaligned terminal nucleotides from poly(A) indicate a polyadenylation site in the known or mapped reference. The requirement of existence of more than or equal to two (≥2) unaligned terminal nucleotides from poly(A) is an additional quality control element, i.e. data filtering, as mere alignment by itself does not guarantee identification of a poly(A) site. Alignment plus existence of more than or equal two (≥2) unaligned terminal nucleotides from poly(A) is sufficient to indicate a polyadenylation site in the known or mapped reference.

The present invention additionally embodies 3′-READS+PAT, which as previously discussed employs an additional poly(A) tail analysis after performing a modified version of 3′-READS+. READS+PAT takes advantage of differential affinities of RNAs with different poly(A) tail lengths to the capture oligonucleotide (e.g. oligo(dT)) molecules to separate RNAs with long and short poly(A) tails from one another. This is an improvement over the method disclosed in Meijer et al. (2007) Nucleic Acids Res 35, e132, hereby incorporated by reference in its entirety, as the present method is based on sequencing and is specific for each poly(A) site. 3′READS+PAT primarily modifies the first “module” of 3′READS+, with an additional step at the end of the second “module” of calculating poly(A) tail length.

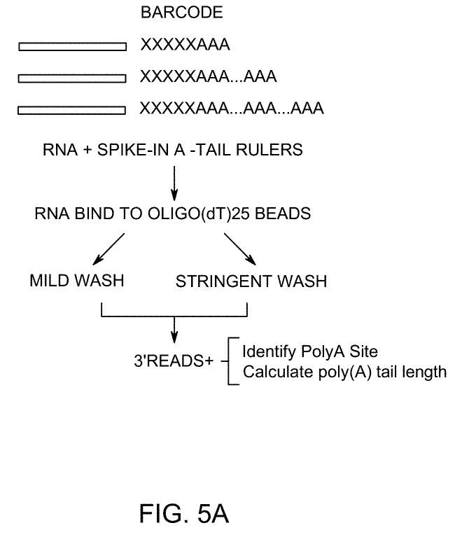

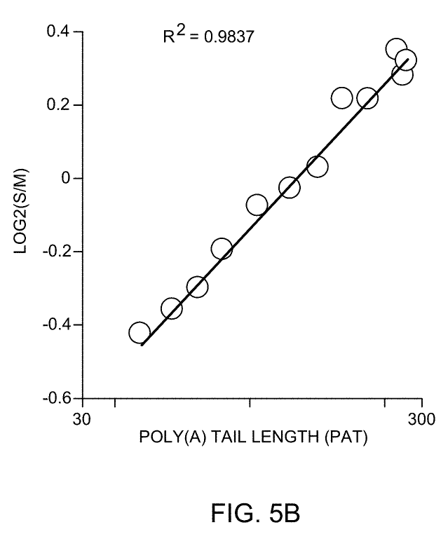

3′READS+PAT is examined in Example 2, FIG. 5, and generally comprises the following steps. First, a sample (e.g. a biological or environmental sample) containing poly(A)+ RNA is obtained. Next, the sample is spiked with a pre-determined quantity of RNAs having identical sequences except for variable lengths of the poly(A) tail; see FIG. 5A for a depiction. These RNAs may be referred to as “barcode” RNAs because their sequence is known and may be used as a control or reference for determining poly(A) tail length of the poly(A)+ RNA present in the sample. The “spiked” sample is then contacted with a capture oligonucleotide, e.g. oligo(dT)₂₅ bound to magnetic beads. Next, either a mild wash (“Mild Wash” sample) or a stringent wash (“Stringent Wash” sample) (see Example 2 for strictly exemplary washes) is applied to the bound poly(A)+ RNA sample to elute poly(A)+ RNA. The conditions of the wash will determine the poly(A) tail length of the resultant poly(A)+ RNA. These steps comprise the modified first “module” of 3′READS+PAT. The second “module” of 3′READS+PAT generally follow the second “module” of 3′READS+, i.e. ligation with a 5′ adapter, use of a chimeric oligonucleotide (CO) according to the present disclosure, incubation/partial digestion with RNase H, elution from the PO, ligation with a 3′ adapter, optionally in the presence of a crowding agent, reverse transcription, amplification, and alignment with a reference, e.g. a reference gene, genome, or database. 3′READS+PAT has an additional final step, however, of calculating poly(A) tail length. This may be done according to the formula set forth in Example 2, which is the log 2(ratio) of the read number from the “Stringent Wash” sample to that from the “Mild Wash” sample, although other formulae are conceivable and should be considered in the scope of the present invention.

3′READS+ offers significant advantages over the prior art, and they relate to several technical features discussed supra. These include, but are not limited to, utilization of antisense oligonucleotides, in particular locked nucleic acids, e.g. locked deoxythymidine (+T) in the PR of the PO, separation of the first ligation step (′5 adapter) from the second ligation step (3′adapter, e.g. 5′ adenylated 3′ adapter), and utilization of a crowding agent during the second ligation step (e.g. PEG). These technical features allow for more comprehensive capture of poly(A)+ RNA throughout the methodology of 3′READS+, greatly improved ligation efficiency, and more thorough elimination of junk RNA leading to better data quality during sequence alignment.

Known methods may utilize DNA/RNA hybrid oligonucleotide containing deoxythymidines (Ts) and uridines (Us) for the chimeric oligonucleotide (“CO”) to remove the bulk of poly(A) tail by RNase H, leaving behind a few As that are annealed to the Us and are thus undigested by the enzyme. An exemplary oligonucleotide of such methods might contain 15-25 U's and 25-35 T's. The terminal A's that are un-alignable to the genome are considered as evidence of the poly(A) tail, allowing identification of genuine poly(A) sites. However, desirable poly(A) protection may be achieved with RNase H at 1/32 U/reaction (FIG. 1A), variation of its concentration by merely 2-fold results in either over or insufficient digestion ( 1/16 and 1/64 U/reaction in FIG. 1A, respectively), indicating that uridines do not give reliable protection of adenosines in RNase H digestion. This problem is not appreciated by the prior art.

While not wishing to be bound by theory, the lack of robustness in protection of As by Us is believed to be caused by interaction between the 14-20 remaining adenosines after the initial round of RNase H digestion and the deoxythymidines in the oligonucleotide, which initiates a second round of RNase H digestion, or indiscriminant digestion of RNA:RNA molecules corresponding to high RNase H concentration. As detailed throughout, one such solution of the present invention is to utilize locked nucleic acids, i.e. locked deoxythymidine instead of uracil or uracil analogs. The PRs of the present invention, particularly utilizing locked deoxythymidine, represent a surprisingly superior technical solution to preventing degradation by RNase H than uracil/uridine or uracil/uridine analogs. A representative LNA/DNA hybrid oligo was designed in Example 1 infra consisting of fifteen consecutive deoxythymidines (T) in the 5′ region and five pairs of alternating locked (+T) and regular (T) deoxythymidines, thus eliminating the need for use of uracil or uracil analogs in the PO, e.g. 5′-T₁₅(+TT)₅-3′ (SEQ ID NO: 3). The inventors discovered by using an oligonucleotide containing 50 Ts (T₅₀) (SEQ ID NO: 7) as a control, that at 0.5 U RNase H/reaction, the highest concentration of RNase H tested, the T₁₅(+TT)₅ (SEQ ID NO: 3) containing CO preserved ˜13 As, whereas the T₅₀ (SEQ ID NO: 7) and T₃₅U₁₅ (SEQ ID NO: 2) oligos led to digestion of 60 As into 3-5 As, representing a substantial increase in quality in the use of locked deoxythymidine to uridines. This result indicated that the T₁₅(+TT)₅ (SEQ ID NO: 3) CO is reliable for protection of the poly(A) RNA from RNase H digestion at surprisingly high RNase H concentration.

It has also discovered that separating the ligation into two distinct steps, a first ligation step and a second ligation step, along with utilization of a crowding agent during the 3′ adapter ligation, greatly improves ligation efficiency and leads to more thorough elimination of junk RNA post digestion by RNase H. The efficiency is marked over known methods, such as having RNA fragments ligated to a 3′ adapter with a truncated T4 RNA ligase II, and then to a 5′ adapter by T4 RNA ligase I in the same reaction tube, an approach often used in small RNA sequencing. Furthermore, the first ligation step of the present invention occurs prior to digestion by RNase H, while the second ligation step occurs in the presence of a crowding agent and post digestion by RNase H.

The present invention embodies kits that may be utilized for modified 3′ region extraction and deep sequencing of polyadenylated RNA to measure RNA abundance and identification of poly(A) site. The kits may contain a chimeric oligonucleotide (CO) as described according to any aspect of this invention, e.g. a CO having a protection region (PR) and a digestion region (DR). The kits may further contain RNase H, ligation adapters, one or more ligases, one or more crowding agents, buffers, reagents for extraction, reagents for precipitation and recovery, reagents for reverse transcription, and/or reagents for amplification (e.g. PCR), and combinations thereof. The kits may contain controls. The kits may contain instructions or directions for use. The kit may be comprised of one or more containers and may also include collection equipment, for example, bottles, bags (such as intravenous fluids bags), vials, syringes, and test tubes. Other components may include needles, diluents and buffers. Usefully, the kit may include at least one container comprising a pharmaceutically-acceptable buffer, such as phosphate-buffered saline, Ringer's solution and dextrose solution. Optionally, the kits of the invention further include software to expedite the generation, analysis and/or storage of data, and to facilitate access to databases. The software includes logical instructions, instructions sets, or suitable computer programs that can be used in the collection, storage and/or analysis of the data. Comparative and relational analysis of the data is possible using the software provided. The kit may be comprised of one or more containers and may also include collection equipment, for example, bottles, bags (such as intravenous fluids bags), vials, syringes, and test tubes. Other components may include needles, diluents and buffers. Usefully, the kit may include at least one container comprising a pharmaceutically-acceptable buffer, such as phosphate-buffered saline, Ringer's solution and dextrose solution. The kit may contain any or all of the following: assay reagents, buffers, probes and/or primers, and sterile saline or another pharmaceutically acceptable emulsion and suspension base. The kits may be used for methods according to the present disclosure, including, but not limited to, identifying poly(A) sites in a reference, e.g. a reference gene, genome, or genomic database, calculating poly(A) tail length, as well as identification of the 3′ end of poly(A)+ RNA encoded in the reference, e.g. gene, genome, or genomic database as well as gene expression analysis, e.g. by determining relative abundance of poly(A) tail containing mRNA in a sample.

“Attached” or “immobilized” as used herein may refer to binding between a support (such as a solid substrate) and a molecule such as an oligonucleotide, or a binding interaction between a ligand and its target. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

A “solid substrate” may be in the form of beads, particles or sheets, a column, an array and may be permeable or impermeable, wherein the surface is coated with a suitable material enabling binding of a target molecule at high affinity. For example, a bead may be coated with strepavidin, and a target molecule bound to biotin will bind to the strepavidin bead with high affinity.

“Array” as used herein may refer to a solid support having a plurality of locations to attach a nucleotide sequence

“Biological sample” as used herein means a sample of biological tissue or fluid that comprises polypeptides and/or nucleic acids. Such samples include, but are not limited to, tissue isolated from animals. Biological samples may also include sections of tissues such as biopsy and autopsy samples, frozen sections taken for histologic purposes, blood, plasma, serum, sputum, saliva, stool, tears, mucus, hair, and skin. Biological samples also include explants and primary and/or transformed cell cultures derived from patient tissues. A biological sample may be provided by removing a sample of cells from an animal, but can also be accomplished by using previously isolated cells (e.g., isolated by another person, at another time, and/or for another purpose), or by performing the methods of the invention in vivo.

As used herein and in the appended claims, the singular forms “a”, “and” and “the” include plural references unless the context clearly dictates otherwise

The term “about” refers to a range of values which would not be considered by a person of ordinary skill in the art as substantially different from the baseline values. For example, the term “about” may refer to a value that is within 20%, 15%, 10%, 9%, 8%, 7%, 6%, 5%, 4%, 3%, 2%, 1%, 0.5%, 0.1%, 0.05%, or 0.01% of the stated value, as well as values intervening such stated values.

Publications disclosed herein are provided solely for their disclosure prior to the filing date of the present invention.

Where a value of ranges is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference in their entireties.

Each of the applications and patents cited in this text, as well as each document or reference, patent or non-patent literature, cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference in their entirety. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

The following non-limiting examples serve to further illustrate the present invention.

### VIII. EXAMPLES

### Example 1—3′READS+

### A. Methods and Materials

### Cells and RNAs Utilized

Human HeLa cells were cultured in high glucose Dulbecco's Modification of Eagle's Medium (DMEM) with 10% fetal bovine serum (Atlanta Biologicals). Total cellular RNA was extracted using the TRIzol reagent (Life Technologies). RNA concentration was measured with NanoDrop 2000 (Thermo Scientific) and RNA quality was examined on an Agilent Bioanalyzer using the RNA 6000 pico kit.

### In Vitro Synthesized RNAs

Plasmids expressing RNAs containing 15, 30, or 60 terminal As (A15, A30, or A60, respectively), named pALL-A15, pALL-A30 or pALL-A60, respectively, were obtained from Bio Scientific Co. Plasmids expressing RNAs containing 5, or 10 terminal As (A5 or A10, respectively) were made by subcloning sequences containing 5 and 10 As into the pALL-A60 plasmid using EcoRI and PvuII sites. All in vitro transcription products of these plasmids were the same except for the poly(A) length. Template for A0 was prepared by cutting the HindIII site right upstream of the A60 sequence in the pALL-A60 plasmid. Radioactively labeled RNAs were synthesized by in vitro transcription with SP6 RNA polymerase (Promega) and linearized plasmids. α-P32 uridine 5′-triphosphate (PerkinElmer) was used for labeling of RNA. RNAs were purified with Micro Bio-Spin P-30 gel columns (Bio-Rad).

### RNase H Digestion Assay

Radioactive A60 RNA was first denatured by heat, captured by biotin-T₃₅U₁₅ (SEQ ID NO: 2) (IDT), biotin-T₅₀ (IDT), or biotin-T₁₅(+TT)₅ (SEQ ID NO: 3) (Exiqon) oligos attached to magnetic beads (Dynabeads MyOne Streptavidin Cl, Life Technologies) at room temperature for 30 min on a rotator, and digested with different concentrations of RNase H (Epicentre) at 37° C. for 30 min. The whole reaction was mixed with an equal volume of 2×RNA loading buffer (95% formamide, 0.02% SDS, 0.02% bromophenol blue, 0.01% xylene cyanol and 20 mM EDTA), incubated at 70° C. for 5 min, and put on a magnetic stand. The supernatant was resolved on an 8% TBE-Urea-polyacrylamide gel. Radioactive signals were analyzed using a phosphor screen (Amersham) and a Typhoon 9400 scanner (Amersham). Image quantification and calculation of molecular weight using molecular size makers were carried out with the ImageJ software.

### RNA Binding Assay

The A60 RNA was mixed with A15, A10, or A5 RNAs, followed by heat denaturation and incubation with the biotin-T₁₅(+TT)₅ oligo attached to magnetic beads (Dynabeads MyOne Streptavidin Cl, Life Technologies) at room temperature for 30 min on a rotator. The beads were then washed three times with buffers containing different concentrations of NaCl and formamide, mixed with 1×RNA loading buffer, heated at 70° C. for 5 min, and put on a magnetic stand. RNA in the supernatant was then analyzed by gel electrophoresis and by autoradiography as described above. The A10 and A15 signals were normalized to the A60 signal in the same lane.

### Adapter Ligation Assays

In vitro transcribed radioactive A30 was captured using oligo(dT)₂₅ beads, dephosphorylated with calf intestinal alkaline phosphatase (NEB) at 37° C. for 45 min, and then phosphorylated with T4 polynucleotide kinase (NEB) at 37° C. for 45 min (on a rotator). RNA was then washed to remove free ATP, and eluted from the beads with nuclease-free H2O. Two types of ligation protocols were tested. In protocol A, a 5′ adenylated 3′ adapter made by the 5′ DNA Adenylation Kit (NEB) was ligated to A30 using T4 RNA ligase II (truncated KQ version, NEB) with or without 15% polyethylene glycol (PEG) 8000 (NEB) at 22° C. for 1 hr. The reaction was then incubated in the same tube with a 5′ adapter, 1 mM ATP and T4 RNA ligase I at 22° C. for 1 hr. In protocol B, A30 was ligated to the 5′ adapter with T4 RNA ligase I (NEB) at 22° C. for 1 hr, in the presence of ATP. The RNA was then captured using oligo(dT)₂₅ magnetic beads (NEB) and eluted with H₂O at 70° C. for 2 min, followed by ligation to the 5′ adenylated 3′ adapter by the T4 RNA ligase I in the presence of 15% PEG 8000. The RNAs in the reactions were then purified by phenol-chloroform extraction, precipitated in ethanol, and examined by gel electrophoresis and by autoradiography as described above.

### 3′READS+

Poly(A)+ RNA in 0.1-15 μg of total RNA was captured using 12 μl of oligo(dT)₂₅ magnetic beads (NEB) in 200 μl 1× binding buffer (10 mM Tris-Cl, pH7.5, 150 mM NaCl, 1 mM EDTA, and 0.05% TWEEN 20) and fragmented on the beads using 1.5 U of RNase III (NEB) in 30 μl RNase III buffer (10 mM Tris-Cl pH8.3, 60 mM NaCl, 10 mM MgCl2, and 1 mM DTT) at 37° C. for 15 min. After washing away unbound RNA fragments with binding buffer, poly(A)+ fragments were eluted from the beads with TE buffer (10 mM Tris-Cl, 1 mM EDTA, pH 7.5) and precipitated with ethanol, followed by ligation to 3 pmol of heat-denatured 5′ adapter (5′-CCUUGGCACCCGAGAAUUCCANNNN, Sigma) (SEQ ID NO: 8) in the presence of 1 mM ATP, 0.1 μl of SuperaseIn (Life Technologies), and 0.25 μl of T4 RNA ligase 1 (NEB) in a 5 μl reaction at 22° C. for 1 hr. The ligation products were captured by 10 pmol of biotin-T₁₅-(+TT)₅ attached to 12 μl of Dynabeads MyOne Streptavidin Cl (Life Technologies). After washing with washing buffer (10 mM Tris-Cl pH7.5, 1 mM NaCl, 1 mM EDTA, and 0.05% TWEEN 20), RNA fragments on the beads were incubated with 0.01 U/μl of RNase H (Epicentre) at 37° C. for 30 min in 30 μl of RNase H buffer (50 mM Tris-Cl pH 7.5, 5 mM NaCl, 10 mM MgCl2, and 10 mM DTT). After washing with RNase H buffer, RNA fragments were eluted from the beads in elution buffer (1 mM NaCl, 1 mM EDTA, and 0.05% TWEEN 20) at 50° C., precipitated with ethanol, and then ligated to 3 pmol of heat-denatured 5′ adenylated 3′ adapter (5′-rApp/NNNGATCGTCGGACTGTAGAACTCTGAAC/3ddC) (SEQ ID NO: 9) with 0.25 μl T4 RNA ligase 2 (truncated KQ version, NEB) at 22° C. for 1 hr in a 5 μl reaction containing 15% PEG 8000 (NEB) and 0.2 μl of SuperaseIn (Life Technologies). The ligation products were then precipitated and reverse transcribed using M-MLV reverse transcriptase (Promega), followed by PCR amplification using Phusion high-fidelity DNA polymerase (NEB) and bar-coded PCR primers for 12-18 cycles (12 cycles for 15 μg input RNA, 13 cycles for 5 μg input, 15 cycles for 1 μg input, and 18 cycles for inputs below 1 μg). RT primers and PCR primers with indexes are described in Table 1 below.

TABLE 1
Adapters and Primers Utilized

| | |
|---|---|
| PCR Primer | AATGATACGGCGACCACCGAGATCTACA |
| | CGTTCAGAGTTCTACAGTCCGA |
| | (SEQ ID NO: 10) |
| PCR Primer | CAAGCAGAAGACGGCATACGAGATCGTGA |
| | TGTGACTGGAGTTCCTTGGCACCCGAGAATTCCA |
| | (SEQ ID NO: 11) |
| RT Forward | CTAGCAGCCTGACATCTTGAGACTTG |
| Primer | (SEQ ID NO: 12) |
| RT Reverse | GCCTTGGCACCCGAGAATTCCA |
| Primer | (SEQ ID NO: 13) |

PCR products were size-selected twice with AMPure XP beads (Beckman Coulter), using 0.6 volumes of beads (relative to the PCR reaction volume) to remove large DNA molecules and an additional 0.4 volumes of beads to remove small DNA molecules. The eluted DNA was selected again with 1 volume of AMPure XP beads to further remove small DNA molecules. The size and quantity of the libraries eluted from the AMPure beads were examined using a high sensitivity DNA kit on an Agilent Bioanalyzer (Agilent). The library concentrations were further measured by qPCR using primers corresponding to 5′ and 3′ end regions of cDNAs. Libraries were sequenced on an Illumina HiSeq 2000 machine (1×50 bases). Raw read numbers are shown in Table 2 below.

TABLE 2
Read Statistics

| Samples | No. of raw reads | No. of PASS reads |
|---|---|---|
| 100 ng HeLa cell total RNA | 17,758,220 | 6,352,086 |
| 200 ng HeLa cell total RNA, | 9,801,509 | 2,389,878 |
| batch 1 | | |
| 200 ng HeLa cell total RNA, | 17,966,592 | 4,764,266 |
| batch 2 | | |
| 400 ng HeLa cell total RNA | 19,670,102 | 5,717,973 |
| 1 μg HeLa cell total RNA, | 9,400,843 | 2,548,297 |
| batch 1 | | |
| 1 μg HeLa cell total RNA, | 16,924,095 | 5,753,272 |
| batch 2 | | |
| 5 μg HeLa cell total RNA, | 13,183,946 | 2,815,806 |
| batch 1 | | |
| 5 μg HeLa cell total RNA, | 16,636,551 | 5,435,961 |
| batch 2 | | |
| 15 μg HeLa cell total RNA | 16,338,443 | 6,126,184 |

### Data Analysis

The sequence corresponding to 5′ adapter was first removed from raw 3′READS+ reads using the cutadapt program. The 5′ random nucleotides and 5′-Ts in the reads were trimmed before the reads were mapped to the human (hg19) genome using Bowtie 2.0 (global mode). Only reads with a mapping quality score (MAPQ) ≥10 were used for further analysis. The trimmed 5′-Ts of each read were then compared to the genomic region downstream of the last aligned position of the read to identify aligned 5′-Ts. The reads with ≥2 non-genomic 5′Ts after this process were called polyA site supporting (PASS) reads. Cleavage sites within 24 nt of each other were clustered into polyA sites. UPM of a transcript with a given poly(A) site was calculated with unique PASS reads, based on 5′ random nucleotides, number of 5′ Ts, and cleavage site location. The 3′READS data were the mouse mixed cell lines Tib75, CMT93, B16, F9, and C2C12. Sequencing quality scores were retrieved using the Biostrings package of Bioconductor.

### B. Results

Efficient Ligation Steps Improve cDNA Yield and Data Quality

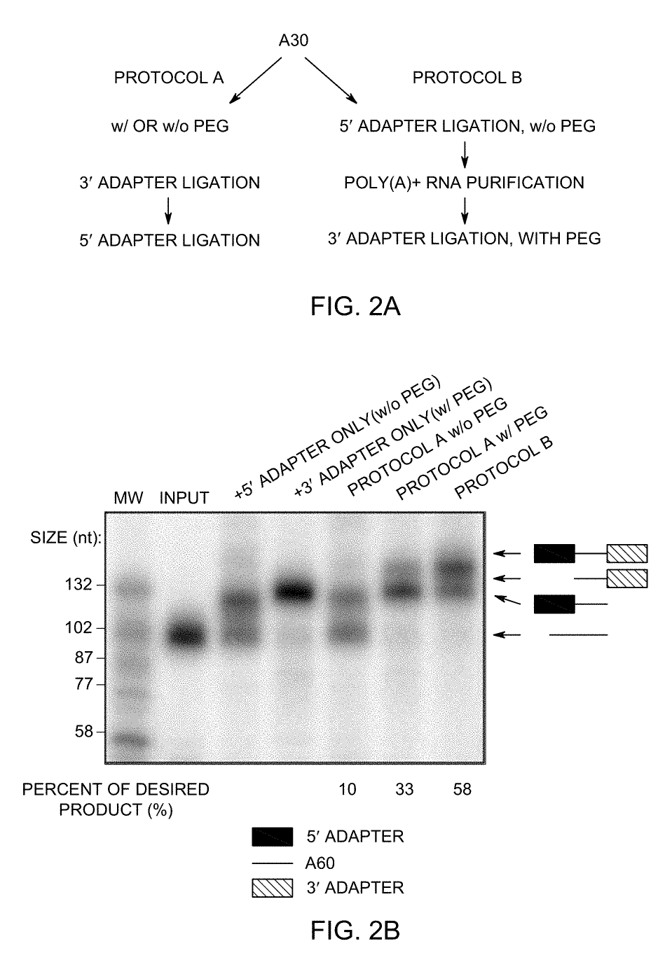

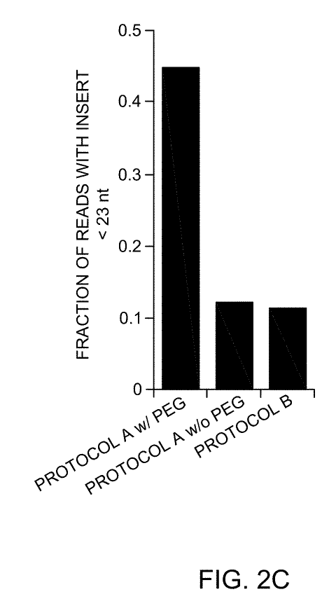

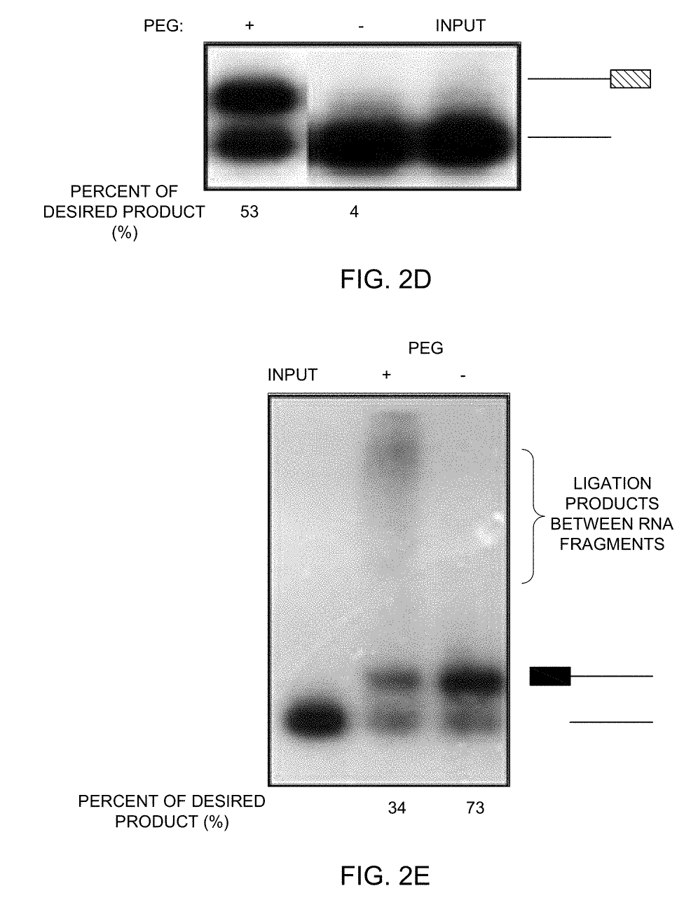

In an effort to improve ligations of 5′ and 3′ adapters separately, it was found that while PEG could significantly stimulate 3′ adapter ligation efficiency by >10-fold FIG. 2D), its enhancement of 5′ adapter ligation was limited (FIG. 2E). In fact, PEG is problematic for 5′ adapter ligation because it also caused concatenation of RNA fragments, leading to a lower amount of desirable products (FIG. 2E). In view of these surprising results, 5′-adapter ligation was performed in the absence of PEG, followed by purification of RNA using oligo(dT) beads to eliminate unused 5′ adapters. Purified RNA was then ligated to the 3′-adapter in the presence of PEG. This new protocol (protocol B in FIG. 2A) resulted in 5.8-fold increase of the amount of desirable product compared to protocol A without PEG (58% vs. 10%) and 1.8-fold increase compared to protocol A with PEG (FIG. 2B). This represents a significant increase over 3′ ligation steps not utilizing a crowding agent such as PEG. Importantly, the fraction of reads with insert size <23 was ˜12%, comparable to protocol A without PEG (FIG. 2C).

### 3′READS+ is Sensitive and Robust

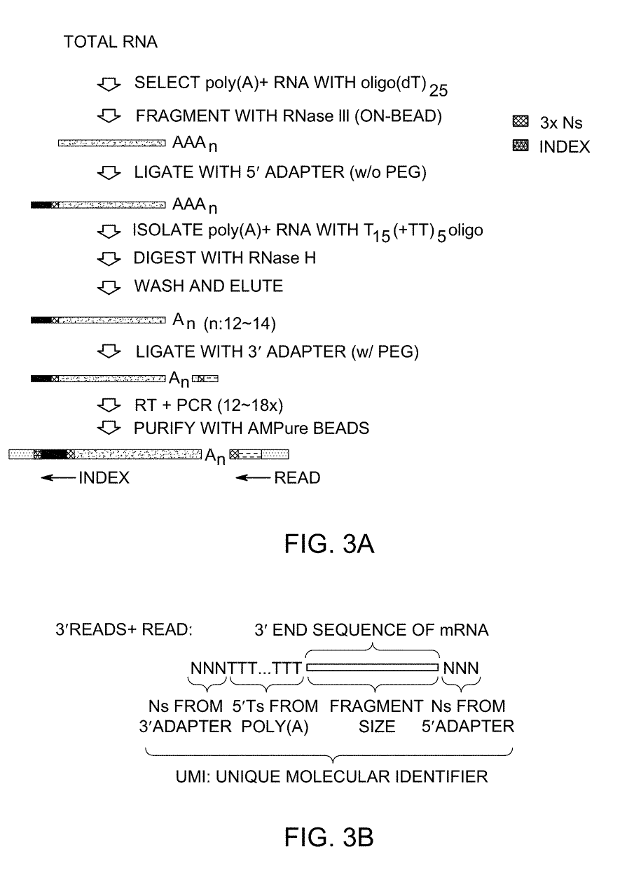

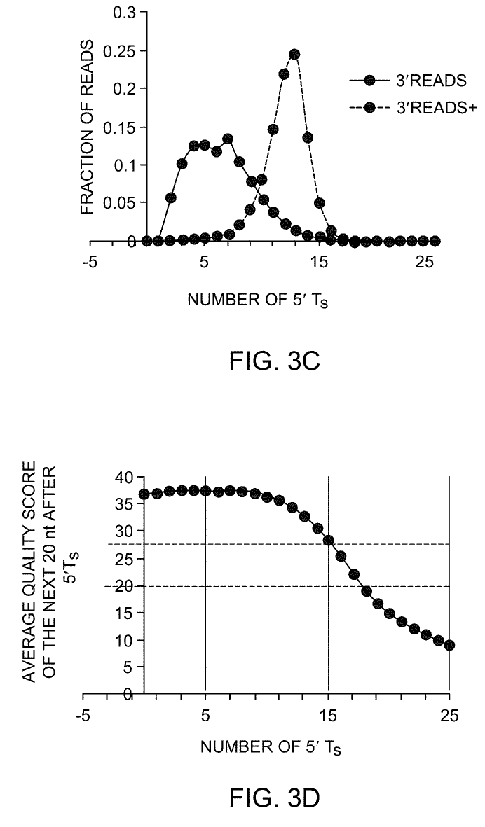

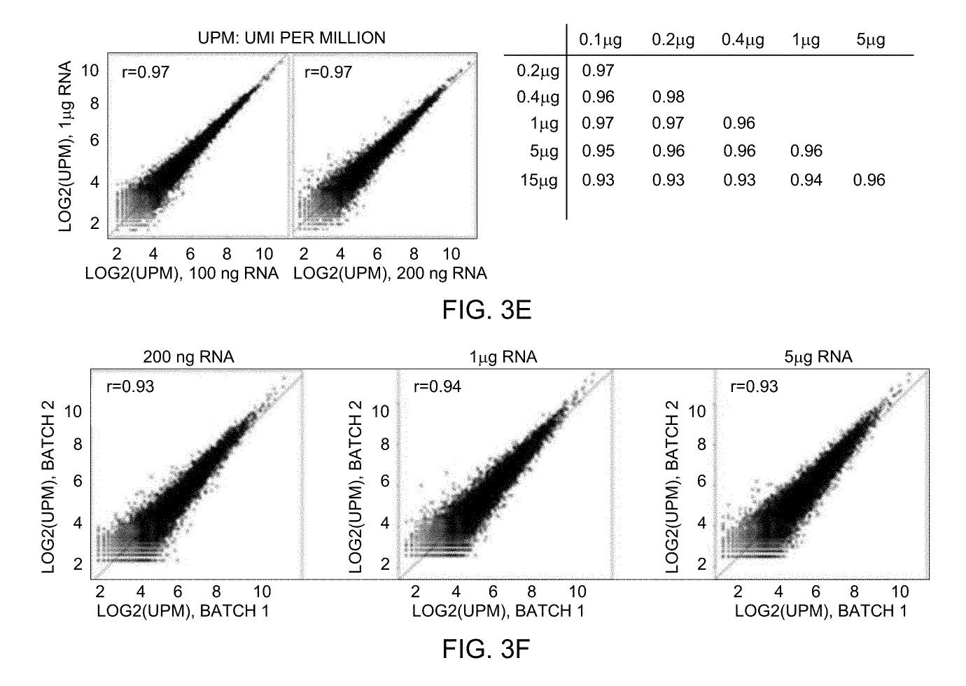

Based on the optimization experiments described above, a new protocol was designed. An exemplary but explicitly non-limiting flowchart of such protocol is illustrated in FIG. 3A. Briefly, poly(A)+ RNA was first selected using oligo(dT)₂₅ beads and fragmented by RNase III on the beads. After washing the beads, poly(A)+ RNA fragments were eluted and ligated to a 5′ adapter (without PEG). The ligation products with a poly(A) tail length >10 nt were then purified using biotin-T₁₅(+TT)₅ (SEQ ID NO: 3) attached to magnetic beads. Unused 5′ adapter were washed away during this step to eliminate ligation between 5′ and 3′ adapters. While the RNAs were on the beads, longer poly(A) tails were trimmed to ˜13 nt by RNase H. This was followed by rigorous washing to discard any RNA fragments that cannot bind to the chimeric oligonucleotide T₁₅(+TT)₅ (SEQ ID NO: 3). After elution, RNA fragments were ligated with a 5′ adenylated 3′ adapter in the presence of PEG. The 5′ and 3′ adapters contained several random nucleotides next to the ligation end to mitigate ligation bias. The ligation products were then reverse transcribed, PCR-amplified (12-18 cycles) with primers containing an index sequence for multiplexing in sequencing, and size-selected using AMPure beads.

The libraries were sequenced from the 3′ adapter region (FIG. 3A), yielding reads beginning with several random Ns derived from the 3′ adapter (three Ns in this study) followed by a run of Ts at the beginning (named 5′Ts) corresponding to the poly(A) tail and a reverse complement sequence to the 3′ end region of an RNA (FIG. 3B). Reads with ≥2 unaligned 5′ Ts after mapping to the genome were called poly(A) site-supporting (PASS) reads. Using HeLa cell RNA, it was found that, consistent with the in vitro result, the number of 5′Ts in PASS reads peaked around 13 nt and below 17 nt for 99% of reads (FIG. 3C), indicating protection of ˜13 As at the 5′-most portion poly(A) tail by the T₁₅(+TT)₅ (SEQ ID NO: 3) oligo. By contrast, the data from an alternative known method utilizing uracil as opposed to locked nucleotides in the protection region (PR) and no improved ligation steps showed a peak around 5 nucleotide (FIG. 3C).

The sequencing quality after the 5′T region was examined using averaged Quality Score (QS) over 20 immediately downstream bases. It was found that sequencing up to fifteen 5′-Ts had little effect on the quality of subsequent bases, with the average QS all >28, a value considered to be high quality (FIG. 3D). The QS dropped below 28 but above 20 (a cutoff for poor quality) after sequencing of sixteen to seventeen 5′Ts (FIG. 3D). By contrast, sequencing of eighteen 5′ Ts led to subsequent bases having QS below 20 (FIG. 3D). This result indicates that using a chimeric oligonucleotide (CO) comprising 5′-T₁₅(+TT)₅-3′ (SEQ ID NO: 3) to generate RNA fragments with peak of ˜13 As and no more than 17 As is potentially an optimal design, maximizing the number of As that can be used for poly(A) site identification and yet not compromising sequencing quality in the subsequent region. Despite 5′-T₁₅(+TT)₅-3′ (SEQ ID NO: 3) being a potentially optimal design, there are many such chimeric oligonucleotides that can be used according to this invention, as discussed throughout this disclosure.

The sensitivity and reproducibility of 3′READS+ was tested using 100 ng, 200 ng, 400 ng, 1 μg, 5 μg and 15 μg total RNAs from HeLa cells. Transcript expression levels were examined between the samples. Because RNA fragments can be over-amplified by PCR, leading to redundant reads, the random sequence (3×Ns) derived from 3′ adapter, the number of 5′ Ts, and the cleavage site location, collectively called unique molecular identifier (UMI), were utilized to identify unique RNA fragments and quantify the expression level of each poly(A) site isoform (FIG. 3B). In addition, if the 5′ adapter region was reached by sequencing (when RNA fragment was short), the RNA fragment size and the random sequence from 5′ adapter were also used as part of UMI (FIG. 3B). UMI per million (UPM) was calculated as the quantitative measure of transcript expression. Comparisons between libraries with different amounts of input RNA showed good consistency, with Pearson's correlation coefficients above 0.95 for all comparisons (FIG. 3E), indicating that 3′READS+ has high sensitivity for input RNA as low as 100 ng at least, and high linearity from 100 ng to 5 μg. In addition, libraries were prepared using the same input RNA but at different times to gauge batch differences. As shown in FIG. 3F, the Pearson correlation coefficients between different batches were above 0.93, indicating low batch effect, thus illustrating that 3′READS+ is sensitive and robust.

### 3′READS+ Identifies A-Stretch Poly(A) Sites

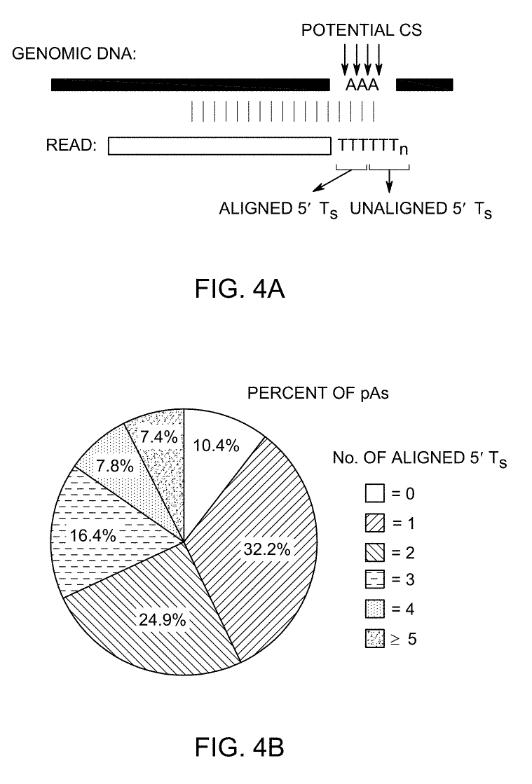

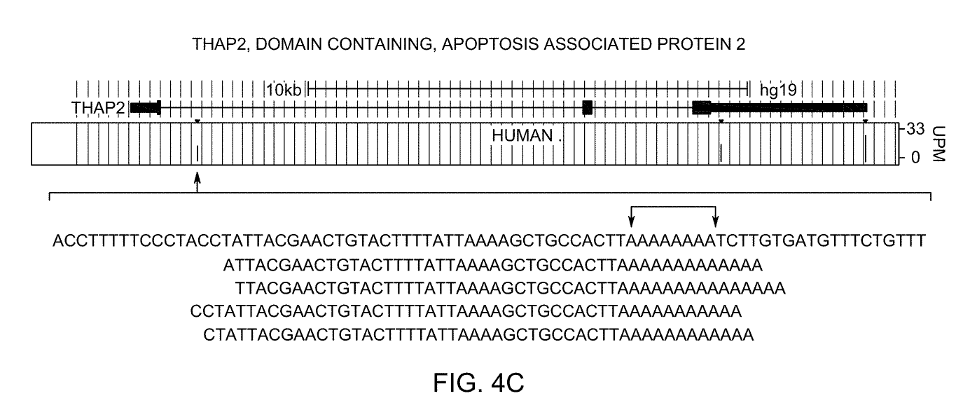

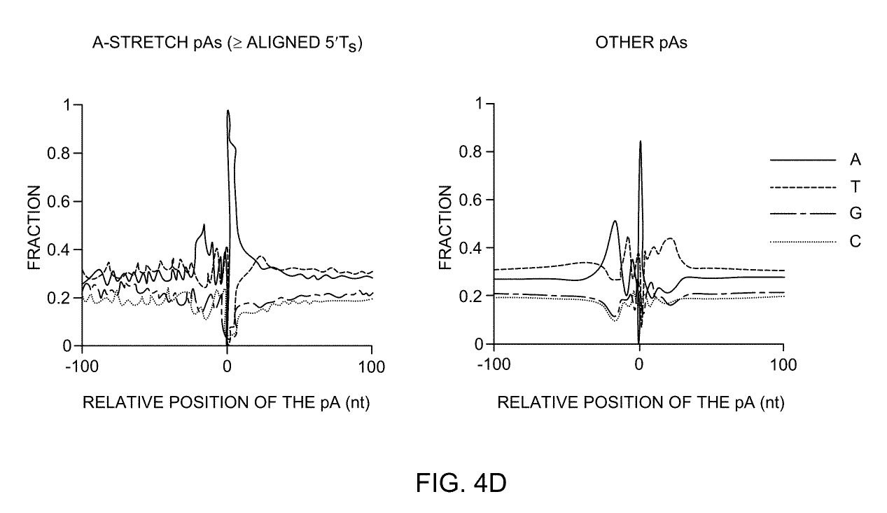

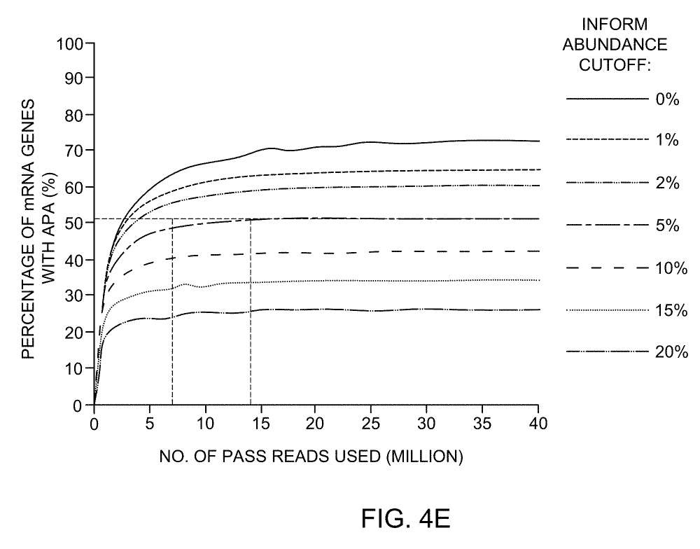

Poly(A) sites can be located within a stretch of As in the genome, making them difficult to identify. For simplicity, these poly(A) sites are called A-stretch poly(A) sites (illustrated in FIG. 4A). They would be discarded from the data generated by oligo(dT)-based 3′ end sequencing, because they could not be distinguished from false sites stemmed from internal priming. Non-oligo(dT)-based methods generate reads with only short As/Ts as poly(A) tail evidence, making them insufficient to identify poly(A) sites located within a long stretch of genomic As. Failure to identify A-stretch poly(A) sites could lead to incomplete mapping of poly(A) sites and inaccurate quantification of APA isoforms or gene expression. Using the HeLa cell data, it was found that about 7.4% of poly(A) sites detected in HeLa cells were within five or more genomic As (FIG. 4B). For some A-stretch poly(A) sites, not all the constituent cleavage sites were within a stretch of poly(A) sites. In these cases, exclusion of A-stretch cleavage sites would lead to partial quantification of poly(A) sites isoform expression. One example of an A-stretch poly(A) site is shown in FIG. 4C, where an intronic poly(A) site of the Thap2 gene is within a stretch of eight genomic As. 3′READS+ reads containing 11-15 5′Ts provided crucial evidence for the identification of this poly(A) site (FIG. 4C). Nucleotide profiles around all A-stretch poly(A) sites (≥5 As) showed upstream A-rich and downstream U-rich peaks similar to those of other poly(A) sites (FIG. 4D), suggesting that A-stretch poly(A) sites are flanked by cis elements similar to other poly(A) sites. Taken together, these data indicate that there exist a sizable fraction of poly(A) sites in the human genome that are located in A-stretch sequences and thus have hitherto been largely overlooked.

### APA in HeLa Cells.

With a total of 42 million (M) PASS reads generated by 3′READS+ with HeLa cell RNAs during the development of the 3′READS+ method (Table 2), it was asked what the APA frequency was for genes expressed in a given type of human cell, like HeLa, an important question that had not been addressed so far. Using random sampling of data from reads from different samples, the APA frequency was assessed with different abundance cutoffs for calling isoforms (FIG. 4E). As expected, more PASS reads identified more genes to display APA, and increasing the isoform relative abundance cutoff led to lower APA rates. For example, with 40M PASS reads, 73% and 26% of genes were found to display APA with 0% and 20% cutoffs, respectively (FIG. 4E). Using relative abundance of 5% to select APA isoforms, a commonly used cutoff value, it was found that the percent of genes expressed in HeLa cells displaying APA plateaued at ˜51% with 14M PASS reads. However, only a slight drop of the rate to 49% when 7M PASS reads were used. Thus, about half of the genes expressed in HeLa cells display APA and >7M PASS reads are needed to have a complete assessment of APA with HeLa cell samples. It is notable, however, these numbers are likely to vary in other cell types when the diversity of transcriptome and APA mechanisms are different.

### Example 2—3′READS+PAT

RNA was first bound to a 25-mer consisting of deoxythymidine (oligo(dT)25) molecules immobilized on magnetic beads and then eluted using buffers with low or high stringency levels for DNA:RNA interactions, named Mild Wash (low stringency) and Stringent Wash (high stringency). The Mild Wash buffer comprised 150 mM NaCl, 10 mM Tris-Cl pH 7.5, 1 mM EDTA and 0.05% (v/v) TWEEN 20, and the Stringent Wash comprised 5% (v/v) formamide, 1 mM NaCl, 10 mM Tris-Cl pH 7.5, 1 mM EDTA and 0.05% (v/v) TWEEN 20. Eluted RNAs were then subject to 3′READS+ processing as described in Example 1 supra, with modifications as discussed herein. This method is illustrated in FIG. 5A. The original RNA sample contained RNA spike-in controls, which are in vitro synthesized RNAs with the same sequences but have different, defined lengthens of the poly(A) tail. Each spike-in control RNA was identified by its barcode located immediately upstream of the poly(A) site. It was found that the log 2(ratio) of the read number from the Stringent Wash sample to that from the Mild Wash sample was a good predictor of poly(A) tail length (FIG. 5B).

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A chimeric oligonucleotide (“CO”) consisting of a protection region (“PR”) and a digestion region (“DR”);
wherein the PR is between 5 and 15 nucleotides in length, the first 5′-nucleotide of the PR is an antisense oligonucleotide which is capable of binding to the poly(A) tail of poly(A)+ RNA, at least one of every three consecutive nucleotides in the PR is an antisense oligonucleotide which is capable of binding to the poly(A) tail of poly(A)+ RNA and protecting the bound poly(A) tail from digestion by RNase H, and the remaining nucleotides in the PR consist of deoxythymidine;
wherein the DR consists of between 5 to 50 deoxythymidines; and
wherein the overall orientation of the CO is 5′-DR-PR-3′.

2. The chimeric oligonucleotide of claim 1, wherein the antisense oligonucleotide comprises at least one of uridine monophosphate, a locked nucleic acid, 2′-O-methyl RNA (OMe), 2′-O-methoxy-ethyl RNA (MOE), N3′-P5′ phosphoramidate (NP), cyclohexene nucleic acid (CeNA), 2-fluoro-arabino nucleic acid (FANA), phosphoroamidate morpholino (PMO), tricyclo-DNA, peptide nucleic acid (PNA), and combinations thereof.

3. The chimeric oligonucleotide of claim 1, wherein the antisense oligonucleotide comprises a locked nucleic acid, and the locked nucleic acid comprises locked deoxythymidine (+T).

4. A kit comprising the chimeric oligonucleotide of claim 1.

5. The use of a kit of claim 4 for one or more of the following:
a) identification of one or more poly(A) sites in a sample; and
b) identification of the 3′ end of a poly(A)+ RNA

6. Use of the kit of claim 4 for analyzing gene expression.

7. A method of identifying a poly(A) site in a reference comprising:
(i) obtaining a sample comprising poly(A)+ RNA;
(ii) contacting the sample with capture oligonucleotide to create isolated poly(A)+ RNA;
(iii) fragmenting the isolated poly(A)+ RNA to create fragmented poly(A)+ RNA;
(iv) eluting the fragmented poly(A)+ RNA from the capture oligonucleotide to create free poly(A)+ RNA;
(v) ligating the free poly(A)+ RNA to a 5′-adapter to create 5′-adapter ligated poly(A)+ RNA;
(vi) contacting the 5′-adapter ligated poly(A)+ RNA with a chimeric oligonucleotide (“CO”) to create CO-bound 5′-adapter ligated poly(A)+ RNA,
wherein the CO consists of a protection region (“PR”) and a digestion region (“DR”), wherein the PR is between 5 and 15 nucleotides in length, the first 5′-nucleotide of the PR is an antisense oligonucleotide which is capable of binding to the poly(A) tail of poly(A)+ RNA, at least one of every three consecutive nucleotides in the PR is an antisense oligonucleotide which is capable of binding to the poly(A) tail of the poly(A)+ RNA and protecting the bound poly(A) tail from digestion by RNase H, and the remaining nucleotides in the PR consist of deoxythymidine,
wherein the DR consists of 5 to 50 deoxythymidines, and
wherein the orientation of the CO is 5′-DR-PR-3′;
(vii) incubating the CO-bound 5′-adapter ligated poly(A)+ RNA with RNase H to partially remove the poly(A) tail of the poly(A)+ RNA to create bound 5′-adapter ligated partially digested poly(A)+ RNA sequencing candidates;
(viii) eluting the bound 5′-adapter ligated partially digested poly(A)+ RNA sequencing candidates from CO to isolate free 5′-adapter ligated partially digested poly(A)+ RNA sequencing candidates;
(ix) ligating the free 5′-adapter ligated partially digested poly(A)+ RNA sequencing candidates to a 3′-adapter to create fully ligated poly(A)+ RNA sequencing candidates;
(x) reverse transcribing the fully ligated poly(A)+ RNA sequencing candidates to create corresponding single-stranded (ss) DNA sequences;
(xi) creating a cDNA library from the corresponding ss DNA sequences; and
(xii) aligning at least one sequence from the cDNA library to a reference, wherein positive alignment against the reference gene or genome and existence of more than or equal to two unaligned terminal nucleotides indicates a poly(A) site in the reference; and
optionally a step of (xiii) calculating the relative abundance of the poly(A)+ RNA to determine a gene expression profile.

8. The method of claim 7, wherein the antisense oligonucleotide comprises at least one of uridine monophosphate, a locked nucleic acid, 2′-O-m20hyl RNA (OMe), 2′-O-methoxy-ethyl RNA (MOE), N3′-P5′ phosphoramidate (NP), cyclohexene nucleic acid (CeNA), 2-fluoro-arabino nucleic acid (FANA), phosphoroamidate morpholino (PMO), tricyclo-DNA, peptide nucleic acid (PNA), and combinations thereof.

9. The method of claim 7, wherein the antisense oligonucleotide comprises a locked nucleic acid, and the locked nucleic acid comprises locked deoxythymidine (+T).

10. The method of claim 7, wherein the poly(A) site identifies the 3′ end of the poly(A)+ RNA in the reference.

11. The method of claim 7, wherein the protection region (PR) of the chimeric oligonucleotide (“CO”) consists of alternating locked/unlocked deoxythymidines.

12. A method of calculating poly(A) tail length comprising:
(i) obtaining a sample comprising poly(A)+ RNA;
(ii) adding a predetermined amount of RNA having identical sequences but with variable poly(A) tail lengths to the sample;
(iii) contacting the sample with a capture oligonucleotide to create isolated poly(A)+ RNA;
(iv) eluting the poly(A)+ containing RNA from the capture oligonucleotide by one of a mild wash or a stringent wash to create free poly(A)+ RNA;
(v) ligating the free poly(A)+ RNA to a 5′-adapter to create 5′-adapter ligated poly(A)+ RNA;
(vi) contacting the 5′-adapter ligated poly(A)+ RNA with a chimeric oligonucleotide (“CO”) to create CO-bound 5′-adapter ligated poly(A)+ RNA,
wherein the CO consists of a protection region (“PR”) and a digestion region (“DR”), wherein the PR is between 5 and 15 nucleotides in length, the first 5′-nucleotide of the PR is an antisense oligonucleotide which is capable of binding to a poly(A) tail of poly(A)+ RNA, at least one of every three consecutive nucleotides in the PR is an antisense oligonucleotide which is capable of binding to the poly(A) tail of the poly(A)+ RNA and protecting the bound poly(A) tail from digestion by RNase H, and the remaining nucleotides in the PR consist of deoxythymidine,
wherein the DR consists of 5 to 50 deoxythymidines, and
wherein the orientation of the CO is 5′-DR-PR-3′;
(vii) incubating the CO-bound 5′-adapter ligated poly(A)+ RNA with RNase H to partially remove the poly(A) tail of the poly(A)+ RNA to create bound 5′-adapter ligated partially digested poly(A)+ RNA sequencing candidates;
(viii) eluting the bound 5′-adapter ligated partially digested poly(A)+ RNA sequencing candidates from CO to isolate free 5′-adapter ligated partially digested poly(A)+ RNA sequencing candidates;
(ix) ligating the free 5′-adapter ligated partially digested poly(A)+ RNA sequencing candidates to a 3′-adapter to create fully ligated poly(A)+ RNA sequencing candidates,
wherein the ligating occurs in the presence of a crowding agent;
(x) reverse transcribing the fully ligated poly(A)+ RNA sequencing candidates to create corresponding single-stranded (ss) DNA sequences;
(xi) amplifying the corresponding ss DNA sequences to create a cDNA library;
(xii) aligning at least one sequence from the cDNA library to a reference, wherein positive alignment against the reference gene or genome and existence of more than or equal to two unaligned terminal nucleotides indicates a poly(A) site in the reference; and
(xiii) calculating poly(A) tail length of the poly(A)+ RNA sequencing candidates, and
optionally a step of (xiv) calculating the relative abundance of the poly(A)+ RNA to determine a gene expression profile.

13. The method of claim 12, wherein the antisense oligonucleotide comprises at least one of a uridine monophosphate, locked nucleic acid, 2′-O-methyl RNA (OMe), 2′-O-methoxy-ethyl RNA (MOE), N3′-P5′ phosphoramidate (NP), cyclohexene nucleic acid (CeNA), 2-fluoro-arabino nucleic acid (FANA), phosphoroamidate morpholino (PMO), tricyclo-DNA, peptide nucleic acid (PNA), and combinations thereof.

14. The method of claim 12, wherein the antisense oligonucleotide comprises a locked nucleic acid, and the locked nucleic acid comprises locked deoxythymidine (+T).

15. The method of claim 12, wherein the poly(A) site identifies the 3′ end of the poly(A)+ RNA in the reference.

16. The method of claim 12 wherein the protection region (PR) of the chimeric oligonucleotide (“CO”) consists of alternating locked/unlocked deoxythymidines.

17. A method to analyze gene expression, the method comprising:
a. obtaining a solution of nucleic acids containing poly(A) sequences;
b. fragmenting said nucleic acids to provide a solution of fragmented nucleic acids;
c. reacting said solution of fragmented nucleic acids with a chimeric oligonucleotide to provide a solution of nucleic acids annealed to the chimeric oligonucleotide and nucleic acids that are not annealed to the chimeric oligonucleotide,
wherein the chimeric oligonucleotide consists of a protection region (“PR”) and a digestion region (“DR”);
wherein the PR is between 5 and 15 nucleotides in length, the first 5′-nucleotide of the PR is an antisense oligonucleotide which is capable of binding to the poly(A) tail of poly(A)+ RNA, at least one of every three consecutive nucleotides in the PR is an antisense oligonucleotide which is capable of binding to the poly(A) tail of poly(A)+ RNA and protecting the bound poly(A) tail from digestion by RNase H, and the remaining nucleotides in the PR consist of deoxythymidine;
wherein the DR consists of between 5 to 50 deoxythymidines; and
wherein the overall orientation of the CO is 5′-DR-PR-3′;
d. removing nucleic acids having short poly (A) sequences with a stringent wash to provide a solution of nucleic acids having long poly (A) sequences annealed to the oligonucleotide;
e. contacting said solution of nucleic acids annealed to said oligonucleotide with an enzyme, wherein said enzyme releases nucleic acids from said oligonucleotide;
f. separating said released nucleic acids to provide a solution of isolated nucleic acids;
g. contacting said solution of purified nucleic acids with a kinase to provide a solution of 5′ phosphorylated nucleic acids;
h. contacting said solution of 5′ phosphorylated nucleic acids with a 3′ adapter, a 5′ adapter, and ligases suitable for ligating said adapters to the 3′ and 5′ ends of the nucleic acids to provide a solution of ligated nucleic acids;
i. contacting said solution with a reverse transcriptase to provide cDNA corresponding to said ligated nucleic acids;
j. amplifying said cDNA corresponding to said ligated nucleic acids by polymerase chain reaction to provide amplified nucleic acids;
k. sequencing said amplified nucleic acids;
l. comparing the sequences of said nucleic acids to the sequence of a reference gene;
m. determining polyadenylation sites in the gene; and
n. calculating the relative abundance of the poly(A)+ RNA to determine a gene expression profile.

18. The method of claim 17, further comprising recording in a computer-readable form detection data indicative of detection of poly (A) sites in a gene.

19. The method of claim 17, wherein said at least one nucleic acid containing a long poly (A) sequence has more than 15 contiguous adenine nucleotides.

20. The method of claim 17, wherein said fragmenting said nucleic acids step comprises fragmenting said nucleic acids with a metal base or a metal ion solution or RNase III, or a combination thereof.
